(19) United States
(12) Patent Application Publication
(10) Pub. No.: US 2011/0287117 A1
(43) Pub. Date: Nov. 24, 2011

(54) Adjuvant Therapy in the Treatment of Gouty Arthritis

(75) Inventor: Naomi Schlesinger, West Orange, NJ (US)

(73) Assignee: UNIVERSITY OF MEDICINE AND DENTISTRY OF NEW JERSEY, Somerset, NJ (US)

(21) Appl. No.: 12/837,258

(22) Filed: Jul. 15, 2010

Related U.S. Application Data
(60) Provisional application No. 61/271,049, filed on Jul. 15, 2009.

(51) Int. Cl. A61K 36/736; A61P 19/02; A61P 19/06

(52) U.S. Cl. 424/735

(57) ABSTRACT

This invention relates to the treatment of gouty arthritis with cherry juice concentrate.

Gouty arthritis is the most common inflammatory arthritis in men over forty. Ongoing reviews of the Cochrane collaboration show that there is little reliable information on which to base treatment decisions in acute gout^{1,2,3}. Acute gouty arthritis is usually treated with non-steroidal anti-inflammatory drugs (NSAIDs) and/or colchicine. NSAIDs decrease pain and swelling in gout as well as the length of the attack. The connection of gout and hyperuricemia with gluttony, overindulgence in food and alcohol and obesity dates from ancient times⁴. Decreasing consumption of meat, seafood and alcoholic beverages as well as increasing consumption of dairy products, cherries and tofu maybe helpful in reducing gouty arthritis⁴. It has been suggested in the popular press and to a lesser extent in the scientific literature that consumption of cherries alleviates arthritic pain and gout due to its anti-inflammatory properties. However, these reports are mostly anecdotal and have not been confirmed in controlled nutrition studies.

It is generally accepted that the clinical manifestations of gout are mediated by inflammation of the joints and it is well documented that monocytes have a role in the onset of acute gout. Monocytes respond to the phagocytosis of MSU crystals by inducing the expression of a number of pro-inflammatory genes, including those encoding interleukin (IL)-1⁵, IL-6⁶, IL-8⁷, IL-11⁸ and TNF-α⁹. The effect of cherries on the serum levels of these cytokines is unknown.

Consumption of cherries and cherry products has been reported previously to be health promoting, particularly in alleviating arthritic pain and gout¹⁰. The effect of cherry consumption on the progression of gout as reflected by the number of gouty attacks and the levels of IL-6 and TNF-α in serum were assessed over a 4 month period.

In this study, we have found that the group treated with cherry juice concentrate had a significant decrease in number of gouty attacks within 4 months of using it. This was not seen in the control group, treated with pomegranate juice. Fifty five percent of patients in the cherry group stopped their NSAIDs after starting using cherry juice due to the decrease in gouty attacks. This suggests that cherries may indeed have anti-inflammatory properties.

It is not known what compounds in cherries might be responsible for these anti-inflammatory actions. Both sweet and tart cherries are rich in antioxidants, including anthocyanins (responsible for red skin and flesh color), catechins, chlorogenic acid, flavonal glycosides and melatonin. Anthocyanins extracted from cherries have shown anti-inflammatory properties, via inhibition of cyclooxygenase (COX) activities^{11,12}. The COX inhibitory activities of anthocyanins from cherries were comparable to those of ibuprofen and naproxen at 10 μM concentrations. Anthocyanins 1 and 2 are present in both cherries and raspberries. The yields of pure anthocyanins 1 and 2 in 100 g of cherries and raspberries were the highest of the fruits tested at 26.5 and 24 mg, respectively. Fresh blackberries and strawberries contained only anthocyanin 2 at a total level of 22.5 and 18.2 mg/100 g, respectively whereas in bilberries, blueberries, cranberries or elderberries do not contain any anthocyanins 1 and 2¹². Thus, cherries contain natural COX 1 and COX 2 inhibitors. In addition, anthocyanins extracted from cherries have shown anti-inflammatory properties, via scavenging of the reactive nitric oxide (NO) radical¹³. Anthocyanins and other phenolics also inhibit NO production in activated macrophages¹⁴ and modulate tumor necrosis factor (TNF-α) secretion¹⁵. Various flavons and flavonols were found to either inhibit or induce TNF-α production¹⁵.

In gouty attacks a network of pro-inflammatory and anti-inflammatory compounds are set into motion by monosodium urate (MSU) crystals¹⁶. Naked MSU crystals are recognized by Toll-like receptors TLR2 and TLR4 which are normally involved in triggering innate host defense responses¹⁷. MSU crystals stimulate synovial cells, monocytes, and neutrophils to produce cytokines and monokines such as TNF-α, interleukin 1 (IL-1), IL-6, IL-8^{5,6,8,16} and IL-18¹⁸. In patients with gouty arthritis the serum levels of C reactive protein (CRP), IL-6, IL-8 and IL-18 were elevated¹⁸ and CXC chemokine receptor-2 ligands were turned on¹⁷. While IL-6 seems to be involved in the pro-inflammatory activity in gout; IL-6 may ameliorate the course of the disease by lowering the SU level. The administration of recombinant IL-6 lowered serum SU level and increased urinary secretion of uric acid¹⁹. Moreover, SU levels were decreased in some patients during acute gouty arthritis while serum IL-6 levels were increased²⁰. The inverse correlation of SU and IL-6 raised the possibility that an inflammatory process plays a role in the increased urinary excretion of uric acid during gouty attacks^{19,20}. The possible role of TNF-α in gout was raised by two case reports in which administration of anti-TNF-α reagents was beneficial in the treatment of severe gouty arthritis^{21,22}.

In this study, no significant changes in the level of either IL-6 or TNF-α were found in gout patients following cherry treatment. In a previous study, cherry consumption by healthy individuals reduced serum CRP but not IL-6²³. The concentration of RANTES (regulated upon activation, normal T-cell expressed, and secreted) and of NO was decreased, while the concentration of a number of other markers of inflammation was not altered by the consumption of cherries^{23,24}. This may be due to the low concentrations of these markers in the healthy participants of this study, low sensitivity of the assay methods used, or the limitation of the effects of cherries to specific inflammatory pathways. It remains possible that other cytokines or chemokines may be involved in amelioration of the clinical symptoms of gout patients.

It has previously been reported, that in healthy individuals cherries provoked a significant decrease in plasma urate over 5 h post dose, whereas other fruits such as strawberries, grapes and kiwifruit, produced no change²⁴. The authors suggested that cherries may exert their urate-lowering effect by increasing the rate of renal glomerular filtration and/or reducing tubular reabsorption. In addition, clinical case reports of three patients with gout showed that consumption of 227 g of cherry products daily for 3 days to 3 months reduced plasma urate to normal levels and alleviated attacks of gouty arthritis²⁵. The mean decrease of SU levels elicited by cherries within 5 hours²⁴ was 14.5%. It is of interest that in the present study while 120 days of cherries had no effect on SU concentration, pomegranate treatment of 5 gout patients reduced SU concentration by 17.5%, although this effect did not reach levels of statistical significance. Since treatment with cherries but not with pomegranates ameliorated the clinical condition of gout patients it seems that the decrease in acute gouty attacks was not associated with a decrease in SU levels in the patients treated with cherry juice.

In conclusion, our study suggests that consumption of cherries reduces acute gouty attacks when taken over a period of 4 months. Cherries may have anti-inflammatory properties and may be a useful adjuvant in the treatment of gouty arthritis.

### Materials and Methods

Patients. Age: Range: 28-75; Mean±SE=56.43±4.10
Disease duration: Range: 3-41; Mean±SE=14.43±3.04
Body mass index (BMI): Range: 24.4-34.4; Mean±SE=30.02±0.84

### Ethnicity: Caucasian 11, Asian 1, Hispanic 1, African American 1

Study design. Eighteen patients were entered into this trial. Four patients dropped out of the study; 2 from the cherry group (n=1 death prior to starting the study; n=1 non compliant), and 2 from the pomegranate group (n=1 severe heartburn, n=1 non compliant). Fourteen patients with crystal proven gout completed an institutional review board (IRB) approved protocol. They were randomized by blindly drawing a folded paper note assigning them to one of the two groups: group A (n=9) received a tablespoon of cherry juice twice daily and group B (n=5) the control group received a tablespoon of pomegranate juice twice daily. Each tablespoon of cherry concentrate is the equivalent of 45-60 cherries and each tablespoon of pomegranate concentrate equals the juice from one pomegranate. Patients continued use of allopurinol, prophylactic colchicine or NSAIDs if they have been taking these drugs prior to initiating the study. Informed consent was obtained from every patient and approval by the IRB was secured prior to the start of enrollment. The parameters recorded at baseline and at 120 days were the number of gouty attacks, medications, serum urate (SU), serum creatinine, serum TNF-α and IL-6 levels. All participant patients received number codes and all evaluations were done blindly.

Cytokine measurement. The levels of TNF-α and IL-6 in patient serum were determined by human cytokines fluorescent bead immunoassay assay kit, Luminex™ from BioSource International (Invitrogen). The assay was performed according to manufacture protocol and analyzed on Luminex™ multiplex reader.
Statistical analysis. The Student's t-test was used to compare the different measured parameters. The significance of changes within each group was analyzed by Student's paired t-test. All P values were 2-tailed and values of less than 0.05 indicate statistical significance.

### Results

Fourteen patients completed this trial. The demographics of the study population are described in the material and methods section. The difference between the number of gouty attacks before and after using cherry juice, assessed by 2-tailed t-test was significant (p<0.05). The results are summarized in Table 1.

TABLE 1
Consumption of cherry juice decreases the number of gouty
attacks and the use of NSAIDs

| | No. of attacks | | No. of attacks | | NSAIDs treatment |
|---|---|---|---|---|---|
| Patient number | before trial | | during study trial | | during trial |
| Group A | | | | | |
| (Cherry) | | | | | |
| 3 | 1 | per month | 0 | per 4 months | Stopped taking |
| | | | | | Celebrex |
| 4 | 3 | per month | 0 | per 4 months | Stopped taking |
| | | | | | Celebrex |
| 5 | 3 | per month | 2 | per month | No change |
| 7 | 1 | per month | 1 | per 2 months | Stopped taking |
| | | | | | Celebrex |
| 8 | 1-2 | per month | 3 | per 4 months | Stopped taking |
| | | | | | indomethacin |
| 10 | 1-2 | per month | 0 | per 4 months | No change¹ |
| 11 | 1 | per year | 0 | per 4 months | No change |
| 14 | 0 | per 4 years | 0 | per 4 months | No change |
| 16 | 2 | per year | 1 | per 4 months | Stopped taking |
| | | | | | indomethacin |
| Group B | | | | | |
| (Pomegranate) | | | | | |
| 1 | 4 | per month | 3 | per month | No change |
| 2 | 1 | per month | 1 | per 4 months | No change |
| 6 | 1-3 | per year | 1 | per 4 months | No change |
| 9 | 2 | per year | No attacks | | No change |
| 13 | 1 | per month | 1 | per month | No change |

¹This patient stopped taking allopurinol.

In the group of 9 patients consuming cherry juice the number of gouty attacks per 4 months at baseline was 4.99±1.53 while on day 120 it was reduced to 1.56±0.88.

Among the 5 patients receiving pomegranate juice the number of gouty attacks per 4 months at baseline was 5.06±2.83 and at day 120 the mean was 3.60±2.20. These data demonstrate that while treatment with cherry juice elicited a significant reduction in the number of gout attacks, treatment with pomegranate juice had no significant effect. Five patients (55%) taking NSAIDs chronically (Celcoxib n=3; indomethacin n=2) discontinued NSAIDs within 60 days of starting cherry juice. None of the patients in the pomegranate group stopped any of their medications. Five patients (55%) in the cherry group were attack free within 4 months of starting cherry juice versus only one patient (20%) in the pomegranate group, however, this patient (patient No. 9) only had 2 attacks in 2 years prior to the start of this trial so it is hard to assess whether the lack of attacks during the 4 months of the trial is due to the consumption of pomegranate juice.

As shown in table 2, The SU levels were only slightly reduced following treatment with either cherry juice (from 8.37±0.82 to 8.17±1.1 mg/dL) or pomegranate juice (from 7.45±1.62 to 6.14±1.07 mg/dL). None of these changes were significant. IL-6 levels were increased at 120 days as compared with baseline in both the cherry group (from 23.29±8.05 to 31.47±9.68 pg/ml) and the pomegranate group (from 17.03±4.27 to 24.00±4.52 pg/ml). However, none of these changes were significant. TNF-α levels were non-significantly reduced in the cherry group (from 34.31±13.82 to 25.21±3.95 pg/ml), while in the pomegranate group the level of TNF-α was non-significantly increased (from 17.36±3.76 to 30.93±5.79 pg/ml). There was also no significant change in serum creatinine levels in either group (data not shown).

TABLE 2
Parameters recorded before and after the trial.

| Parameter | | Day 0, Mean ± SE | Day 120, Mean ± SE |
|---|---|---|---|
| Serum Urate | Group A | 8.37 ± 0.82 | 8.17 ± 1.1 |
| (mg/dL) | Group B | 7.45 ± 1.62 | 6.14 ± 1.07 |
| IL-6 | Group A | 34.50 ± 6.47 | 49.54 ± 9.5 |
| (pg/ml) | Group B | 22.98 ± 0.00 | 35.8 ± 0.00 |
| TNF-α | Group A | 36.54 ± 7.92 | 21.76 ± 0.96 |
| (pg/ml) | Group B | 11.62 ± 0.89 | 30.69 ± 1.83 |

In another retrospective study, the use of cherry juice concentrate was studied.

The goal of this study was to record whether cherry juice concentrate ingested daily was helpful in the treatment of gout patients.

### Method:

We performed a retrospective chart review of patients seen in our clinic between Jul. 1, 2004 and May 1, 2009 with a primary or secondary diagnosis of gouty arthropathy or Gout NOS, diagnosis codes 274.0 or 274.9. The difference between the number of gout attacks before and after Tart cherry juice ingestion was assessed by paired 2-tailed t-test.

### Results:

We identified 24 patients with MSU proven gout that ingested tart cherry juice concentrate (Bronwood Acers) (a tablespoon twice daily). Duration of treatment was ≧4 months (n=24). 11 of 24 (45%) patients were not on urate lowering therapy (ULT). The average SU in patients not on ULT (n=9) was 9.2 mg/dl, while the average SU of patients on allopurinol (n=13) was 6.2 mg/dl. 12 of the entire group of patients (50%) and 4 (36%) of the sub-group of patients who were not on ULT were attack-free at 4-6 months of using cherry juice concentrate. The average SU among all patients who were attack-free was 7.8 mg/dl. A 50% or greater reduction in acute gouty attacks was seen in 22 (92%) of patients. The mean number of acute gout attacks in 11 patients not on ULT was 3.55±1.01 (mean±S.E.) prior to cherry juice ingestion as compared with 1.27±0 541 after 4-6 months of juice ingestion (p=0.004). A similar highly significant effect was obtained with 13 patients who were on. ULT treatment. Cherry juice ingestion decreased the number of acute gouty attacks from 3.54±0.896 to 0.615±0.311 (p=0.0067).

No significant effect of Tart cherry juice concentrate was noted on the SU and creatinine levels in gout patients.

### CONCLUSION

Patients ingesting tart cherry juice concentrate daily for ≧4 months had a highly significant reduction in the number of acute gouty flares. A 50% or greater reduction in acute gouty attacks was seen in. 92% of patients. 36% of patients not on ULT were attack-free at 4-6 months of ingesting cherry juice despite an average SU of 7.8 mg/dl. This study suggests that cherries may have anti-inflammatory properties. Prophylaxis with tart cherry juice concentrate should be considered as an adjunct to ULT.

### REFERENCES

1. Schlesinger N, Baker D G, Schumacher H R, Jr. Gout: How well have diagnostic tests and therapies been evaluated? Current Opinion in Orthopedics 2000; 11:71-76.
2. Schlesinger N, Baker D G, Schumacher H R, Jr. Gout: Can management be improved? Current Opinion in Rheumatology 2001; 13(3):240-244.
3. Schlesinger N, Schumacher H R, Jr. Gout: How much of what we do is evidence based? In: Evidence based Rheumatology. Tugwell P et al. Editors. First Edition. 2003:65-95. British Medical Journal Publishers.
4. Schlesinger N. Dietary factors and hyperuricaemia. Current Pharmaceutical Design 2005:11(32):4133-138.
5. Di Giovine F S, Malawista S E, Nuki G, Duff G W. Interleukin 1 (IL 1) as a mediator of crystal arthritis: stimulation of T cell and synovial fibroblast mitogenesis by urate crystal-induced IL-1. J Immunol 1987; 38:3213-3218.
6. Guerne P A, Terkeltaub R, Zuraw B, Lutz M. Inflammatory microcrystals stimulate interleukin-6 production and secretion by human monocytes and synoviocytes. Arthritis Rheum 1989; 32:1443-1452.
7. Terkeltaub R, Zachariae C, Santoro D, Martin J, Peveri P, Matsushima K. Monocyte-derived neutrophil chemotactic factor/interleukin-8 is a potential mediator of crystal-induced inflammation. Arthritis Rheum 1991; 34:894-903.
8. Trontzas P, Kamper E F, Potamianou A, Kyriazis N C, Kritikos H, Stavridis J. Comparative study of serum and synovial fluid interleukin-11 levels in patients with various arthritides Clinical Biochemistry 1998; 30: 673-679.
9. Di Giovine F S, Malawista S E, Thornton E, Duff G W. Urate crystals stimulate production of tumor necrosis factor alpha from human blood monocytes and synovial cells. Cytokine mRNA and protein kinetics, and cellular distribution. J Clin Invest 1991; 87:1375-1381.
10. Blau L W. Cherry diet control for gout and arthritis. Tex Rep Biol Med 1950; 8:309-311.
11. Wang H B, Nair M G, Strasburg G M, Chang Y C, Booren A M, Gray J I, DeWitt D L. Antioxidant and anti-inflammatory activities of anthocyanins and their aglycon, cyanidin, from tart cherries. J Nat Prod 1999; 62:294-206.
12. Seeram N P, Momin R A, Nair M G, Bourquin L D. Cyclooxygenase inhibitory and antioxidant cyanidin glycosides in cherries and berries. Phytomedicine 2001; 8:362-369.
13. Vanacker S A B E, Tromp M N J L, Haenen G R M M, Vandervijgh W J F, Bast A. Flavonoids as scavengers of nitric-oxide radical. Biochem Biophys Res Commun 1995; 214:755-759.
14. Wang J, Mazza G. Inhibitory effects of anthocyanins and other phenolic compounds on nitric oxide production in LPS/IFN-gamma-activated RAW 264.7 macrophages. J Agric Food Chem 2002; 50:850-857.
15. Wang J, Mazza G. Effects of anthocyanins and other phenolic compounds on the production of tumor necrosis factor alpha in LPS/IFN-gamma-activated RAW 264/7 macrophages. J Agric Food Chem 2002; 50:4183-4189.
16. Liu-Bryan R, Terkeltaub R. Evil humors take their toll as innate immunity makes gouty joints TREM-ble. Arthritis Rheum 2006; 54:383-386.
17. Liu-Bryan R., Scott P, Sydlaske A., Rose D M, R. Terkeltaub R. Innate immunity conferred by Toll-like receptors 2 and 4 and myeloid differentiation factor 88 expression is pivotal to monosodium urate monohydrate crystal-induced inflammation. Arthritis Rheum 2005; 52: 2936-2946.
18. Inokuchi T, Moriwaki Y, Tsutsui H, Yamamoto A, Takahashi S, Tsutsumi Z, Ka T, Nakanishi K, Yamamoto T Plasma interleukin (IL)-18 (interferon-gamma-inducing factor) and other inflammatory cytokines in patients with gouty arthritis and monosodium urate monohydrate crystal-induced secretion of IL-18. Cytokine 2006; 33:21-27.
19. Tsutani H, Yoshio N, Ueda T Interleukin 6 reduces serum urate concentrations. J Rheumatol 2000; 27:554.
20. Urano W, Yamanaka H, Tsutani H, Nakajima H, Matsuda Y, Taniguchi A, Hara M, Kamatani N. The inflammatory process in the mechanism of decreased serum uric acid concentrations during acute gouty arthritis. J Rheumatol 2002; 29:1950-1953.
21. Tausche A K, Richter K, Grassier A, Hansel S, Roch B, Schroder H E Severe gouty arthritis refractory to anti-inflammatory drugs: treatment with anti-tumor necrosis factor alpha as a new therapeutic option. Arm Rheum Dis. 2004; 63:1351-1352.
22. Fiehn C, Zeier M. Successful treatment of chronic tophaceous gout with infliximab (Remicade). Rheumatol Int 2006; 26:274-276.
23 Kelly D S. Rasooly K R, Jacob, R A, Kader A A, Mackey B E. Consumption of Bing Sweet Cherries Lowers Circulating Concentrations of Inflammation Markers in Healthy Men and Women. Journal of Nutrition 2006; 136:981-986.
24 Jacob R A, Spinozzi G M, Simon V A, Kelley D S, Prior R L, Hess-Pierce B, Kader A A. Consumption of Cherries Lowers Plasma Urate in Healthy Women. Journal of Nutrition 2003; 133:1826-1829.
25 Johnson R J, Rideout B A. Uric acid and diet-insights into the epidemic of cardiovascular disease. N Eng J Med 2004; 350:1071-1073.

1. A method to reduce the number of gouty arthritic attacks in a patient suffering from gout which comprises administering to a therapeutically effective amount of cherry concentrate.
